(19) United States
(12) Patent Application Publication
(10) Pub. No.: US 2006/0084922 A1
(43) Pub. Date: Apr. 20, 2006

(54) Device for securing a blood vessel cannula to a body

(76) Inventor: Ruark Botha, Universitas Ridge, Bloemfontein (ZA)

Correspondence Address: MERCHANT & GOULD PC, P.O. BOX 2903, MINNEAPOLIS, MN, 55402-0903, US

(21) Appl. No.: 10/526,857

(22) PCT Filed: Sep. 4, 2003
(86) PCT No.: PCT/ZA03/00128

§ 371 Date: Sep. 28, 2005

(30) Foreign Application Priority Data
Sep. 4, 2002 (ZA) 2002/7083

(51) Int. Cl. A61M 5/32

(52) U.S. Cl. 604/174; 128/DIG. 6

(57) ABSTRACT

The invention provides for a device for securing a portion of a blood vessel cannula to a human being or animal body, the device including a retaining means shaped and dimensioned to retain said cannula portion that extends exterior the body surface in a fixed position on the body; and an attachment means for removable attachment of the device to the skin thereby inhibiting movement of the cannula. The invention also provides for a method of securing a blood vessel cannula to a human being or animal body

### FIELD OF THE INVENTION

The invention is in the field of medical accessories, specifically devices used in the securing of a blood vessel cannula to the skin

### BACKGROUND TO THE INVENTION

Once a cannula has been successfully inserted into a blood vessel, be it intravenous or intra-arterial, it is Imperative that the cannula not be dislodged. Traditional methods require the care giver to pre-cut a plaster or use one of the newer self-adhesive plasters that require the care giver to use both hands. The inserted cannula is thus left and any movement of the patient can cause the cannula to be dislodged. It is also often difficult to secure the cannula and administration tube under one dressing and can thus lead to wastage.

A need exists for a device that can secure a cannula, is easy to use and can be applied with one hand while the other hand is used to stabilize the cannula.

### SUMMARY OF THE INVENTION

According to an aspect of the invention, there is provided a device for securing a portion of a blood vessel cannula to a human being or animal body, the device including;

a retaining means shaped and dimensioned to retain said cannula portion that extends exterior the body surface in a fixed position on the body; and

an attachment means for removable attachment of the device to the skin thereby inhibiting movement of the cannula.

The retaining means may be a deformable receptacle.

The retaining means may be a resiliently deformable receptacle.

The receptacle may include a first open position and a second closed position. When in a closed position, the receptacle may have a frusto-conical shape.

The receptacle may include a locking means configured to lock and maintain the receptacle in a closed position.

The locking means may include a clip or a clasp.

The receptacle may include a recess configured to accommodate a rim on an end of the cannula.

The receptacle may include a viewing port that permits a user to monitor for leakage or dislodgement.

The receptacle may be translucent, thereby permitting inspection of the underlying area and cannula.

The receptacle may be in the form of a clamp.

The attachment means may be in the form of a wing adjacent the receptacle. The wing may be a pliable wing.

A surface of the wing may include on at least a part thereof, an adhesive for attachment to the skin. The adhesive part may be covered by a peel off cover layer which prior to use prevents attachment to other objects. The peel off layer may include a tab configured to facilitate the removal of said layer when the device is to be used. Typically, the adhesive would be of the hypoallergenic type and water soluble.

The device may include a securing means for securing an administration tube that may be attached to the cannula. The securing means may include protrusions. The tube may be guided between said protrusions thereby to inhibit displacement thereof. Typically, the tube is guided between the protrusions before the wings are attached to the skin

It is to be appreciated from this specification, that the receptacle may be configured to accommodate a cannula attached to an administration tube where the tube has been screwed onto the cannula, or where the tube has been fitted into the cannula.

It is further to be appreciated from this specification, that the retaining means may be of unitary moulding with the cannula and may therefore not require the retaining means to be attached to the cannula after insertion of said cannula in the blood vessel.

According to another aspect of the invention, there is provided a method of securing a portion of a blood vessel cannula to a human being or animal body, said method including;

retaining said cannula portion that extends exterior the body surface in a receptacle in a fixed position on the body; and

inhibiting movement of the receptacle by removable attachment thereof to the skin.

Retaining the cannula may be by means of a resiliently deformable receptacle.

Locking the receptacle in a closed position may be by means of a clip or clasp.

Attaching the device to the skin may be by way of a pliable wing adjacent the receptacle that has on a surface of the wing an adhesive configured to be attached to the skin.

Securing an administration tube attached to the cannula may be by way of protrusions. The tube may be guided between said protrusions before attachment to the skin.

### DETAILED DESCRIPTION OF DRAWINGS

The invention will now be described, by way of non-limiting example, with reference to the accompanying diagrammatic drawings wherein:

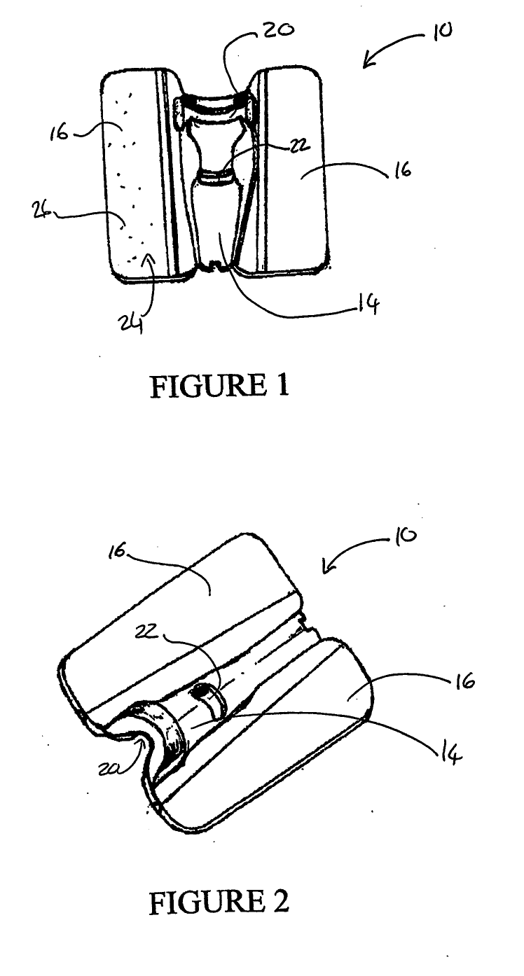

FIG. 1 shows an inferior view of a device for securing a portion of a blood vessel cannula to a human being or animal body.

FIG. 2 shows a top view of the device.

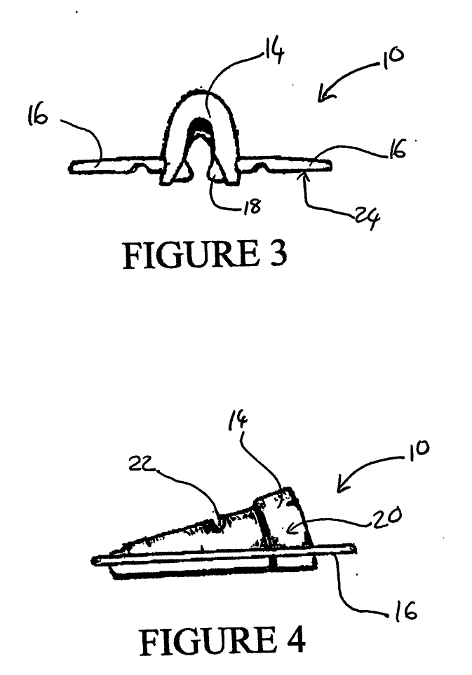

FIG. 3 shows a posterior view of the device.

FIG. 4 shows a side view of the device.

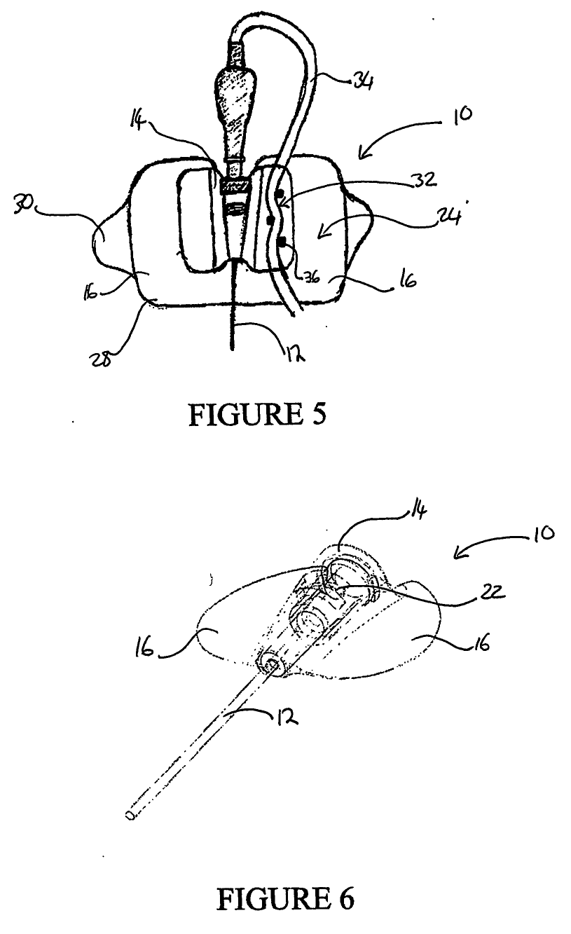

FIG. 5 shows a different embodiment of the invention.

FIG. 6 shows the device as in use, with a part of the cannula being visualized through a transparent retaining means.

In the drawings, reference numeral 10 generally refers to a device for securing a portion of a blood vessel cannula to a human being or animal body.

A device 10 for securing a portion of a blood vessel cannula 12 to a human being or animal body (not shown), the device including a retaining means 14 shaped and dimensioned to retain said cannula 12 portion that extends exterior the body surface in a fixed position on the body and an attachment means 16 for removable attachment of the device 10 to the skin thereby inhibiting movement of the cannula.

In the preferred embodiment, the retaining means 14 is a resiliently deformable receptacle that is capable of clamping a part of a cannula 12.

The receptacle 14 has a first open position (FIGS. 1 to 5) and second closed position (FIG. 6). The receptacle 14 has a frusto-conical shape in the closed position.

The receptacle 14 has a clip 18. Once the receptacle 14 has been closed around a part of the cannula 12, the clip 18 keeps the receptacle in a closed position.

The receptacle 14 has a recess 20 configured to accommodate a rim (not shown) on an end of the cannula 12.

The receptacle 14 includes a viewing port 22. A connection between the cannula 12 and an administration line can be viewed through said port 22. The port 22 therefore allows a user to inspect for leakage and dislodgement.

The receptacle 14 is translucent. The translucency allows for inspection of the underlying area (not shown) and cannula 12.

The attachment means 16 are pliable wings.

A surface 24 of the wings 16 are covered by an adhesive layer 26. A tabbed cover 28 prevents attachment to other objects. The cover 28 is removed by tugging on the tab 30.

In the embodiment shown in FIG. 5, the device 10 includes a securing means 32 for securing an administration tube 34 that is attached to the cannula. The securing means 32 includes protrusions 36. The tube 24 is woven between said protrusions 36 before attaching the wing 16 to the skin.

1. A device for securing a portion of a blood vessel cannula to a human being or animal body, the device including; a retaining means shaped and dimensioned to retain said cannula portion that extends exterior the body surface in a fixed position on the body; and an attachment means for removable attachment of the device to the skin thereby inhibiting movement of the canula.

2. A device as claimed in claim 1, wherein the retaining means is a deformable receptacle.

3. A device as claimed in claim 2, wherein the receptacle includes a first open position and a second closed position.

4. A device as claimed in claim 3, wherein the receptacle has a FRUSTO-CONICAL shape in the closed position.

5. A device as claimed in claim 3, wherein the receptacle includes a locking means configured to lock and maintain the receptacle in a closed position.

6. A device as claimed in claim 1, wherein the retaining means includes a recess configured to accommodate a rim on an end of the canula.

7. A device as claimed in claim 1, wherein the retaining means includes a viewing port that permits a user to monitor for leakage and dislodgement.

8. A device as claimed in any one of claims 1 to 7, wherein the retaining means is translucent and thereby permits inspection of the underlying skin area and canula.

9. A device as claimed in claim 1, wherein the retaining means is in the form of a clamp.

10. A device as claimed in claim 1, wherein the attachment means is in the form of a wing.

11. A device as claimed in claim 10, wherein a surface of the wing includes on at least a part thereof, an adhesive for attachment to the skin.

12. A device as claimed in claim 11, wherein the adhesive part is covered by a peel off cover layer.

13. A device as claimed in claim 12, wherein the peel off layer includes a tab configured to facilitate the removal of said layer before use.

14. A device as claimed in claim 1, wherein the device includes a securing means for securing an administration tube attached to the canula.

15. A device as claimed in claim 14, wherein the securing means includes protrusions that are configured to guide the tube and inhibit displacement thereof.

16. A method of securing a portion of a blood vessel cannula to a human being or animal body, said method including; retaining said cannula portion that extends exterior the body surface in a receptacle in a fixed position on the body; and inhibiting movement of the receptacle by removable attachment thereof to the skin.

17. A method of securing a BLOOD VESSEL CANNULA as claimed in claim 16, wherein the retaining of the cannula is by means of a resiliently deformable receptacle that has open and closed positions.

18. A method of securing a blood vessel cannula as claimed in claim 16, wherein the locking of the receptacle in a closed position is by means of a clip.

19. A method of securing a blood vessel cannula as claimed in claim 16, wherein the inhibiting of movement is by way of a pliable wing that has on a surface of the wing an adhesive configured to be removably attached to the skin.

20. A method of securing a blood vessel cannula as claimed in claim 16, wherein securing of an administration tube attached to the CANNULA is by way of guiding the tube between protrusions located on a surface of the device.

21. A device as claimed in claim 1, substantially as herein described and illustrated.

22. A method as claimed in claim 16, substantially as herein described and illustrated

23. A new device or a new method substantially as herein described.
